(19) United States
(12) Patent Application Publication
(10) Pub. No.: US 2025/0345188 A1
(43) Pub. Date: Nov. 13, 2025

(54) PROSTHETIC FOOT AND ANKLE SYSTEM WITH DAMPER

(71) Applicant: Motion Control, Inc., Salt Lake City, UT (US)

(72) Inventors: Edwin Kay Iversen, Salt Lake City, UT (US); Gregory James Jacobs, Salt Lake City, UT (US); Jeffery David Christenson, West Valley City, UT (US); Carter J. Greene, West Valley City, UT (US)

(73) Assignee: Motion Control, Inc., Salt Lake City, UT (US)

(21) Appl. No.: 19/278,484

(22) Filed: Jul. 23, 2025

Related U.S. Application Data
(63) Continuation of application No. 17/507,532, filed on Oct. 21, 2021.

(51) Int. Cl. A61F 2/66; A61F 2/50; A61F 2/74

(52) U.S. Cl. CPC A61F 2/6607; A61F 2/744; A61F 2/748; A61F 2002/5006; A61F 2002/5016; A61F 2002/5079; A61F 2002/6642

(57) ABSTRACT

Technology is described to provide a foot/ankle prosthesis for individuals with transfemoral limb loss. This technology is able to store and release energy and thus individuals or patients who are using the foot/ankle prosthesis are able to expend less energy when walking or running. The device or system can include a prosthetic foot/ankle system with a linear or rotary hydraulic damper such that the hydraulic damper is attached to dynamic energy storing spring elements. The axis of rotation of the system can be near to that of an intact human ankle. The system can utilize spring elements based on the vertical displacement of the center of pressure of an intact normal foot. The system can also provide user adjustable heel height.

### BACKGROUND

Prostheses (or prosthetics) are artificial devices that replace body parts (e.g., fingers, hands, arms, legs, feet, toes, etc.). Generally, prostheses may be used to replace body parts lost by injury, disease or missing from birth.

In one example, an intact human foot connected at an ankle travels through stance and swing phases of a gait cycle during each stride of motion, whether the motion involves walking, jogging, or running. By adjusting the stiffness and damping characteristics of a prosthetic foot and ankle mechanism, the springiness of natural human foot and the corresponding natural human joints may be mimicked, thereby optimizing the prosthesis for the desired motion of the wearer. However, the characteristics that are desired to store and release energy appropriately for walking tend to oppose those best suited for fast walking and running.

### BRIEF DESCRIPTION OF THE DRAWINGS

FIG. 1 illustrates an example of a prosthetic foot and ankle system that uses a linear hydraulic cylinder and energy storing springs.

FIG. 2 illustrates an example of a prosthetic foot and ankle system that utilizes a rotary hydraulic damper and dynamic energy storing spring elements.

FIG. 3 illustrates a schematic of an example fluid flow configuration for a hydraulic cylinder of the foot/ankle system.

### DETAILED DESCRIPTION

Reference will now be made to the examples illustrated in the drawings, and specific language will be used herein to describe the same. It will be understood that no limitation of the scope of the technology is thereby intended. Alterations and further modifications of the features illustrated herein, and additional applications of the examples as illustrated herein, which would occur to one skilled in the relevant art and having possession of this disclosure are to be considered within the scope of the description.

A technology is described that provides a foot and/or ankle prosthesis for individuals with lower limb loss. This technology is able to store and release energy and thus individuals or patients who are using the foot and/or ankle prosthesis are able to expend less energy when walking or running.

The biomimetic prosthetic foot/ankle described herein has the following configurations that may improve the use of a prosthetic ankle/foot for individuals using a prosthetic limb. The device or system can include a prosthetic foot/ankle system with a linear or rotary hydraulic damper such that the hydraulic damper is attached (e.g., rigidly) to dynamic energy storing spring elements. The axis of rotation of the device or system can be estimated to be near to an axis of rotation of an intact human ankle, which may provide better biomimetic function.

The system can utilize spring elements that are curved or have a curved surface (e.g., to provide a fulcrum) based on the vertical displacement of the center of pressure of an intact normal foot. One such spring element may be an energy storing spring that is a leaf spring. The system can further provide user adjustable heel height using a system to provide user adjustable heel height and adaptation to inclines. For example, the system can also provide a user or amputee with an adjustable heel height using an adjustable sliding yoke.

The system may have an adjustable stiffness toe-lift spring to lift the toe of the foot/ankle system rapidly after toe-off to reduce stumbling and hip hiking. The system can allow a user to adjust both dorsiflexion and plantar flexion resistance independently to vary heel strike hydraulic shock absorption and avoid foot slap at the foot flat position.

This technology has a plurality of configurations. In one configuration, the system can utilize a linear hydraulic damper or linear hydraulic cylinder. In a second configuration, the system can utilize a rotary hydraulic damper or rotary hydraulic damping mechanism. A description of configurations that can utilize a linear hydraulic cylinder are provided but the discussion of the linear hydraulic damping may apply to the rotary hydraulic damping system and vice versa.

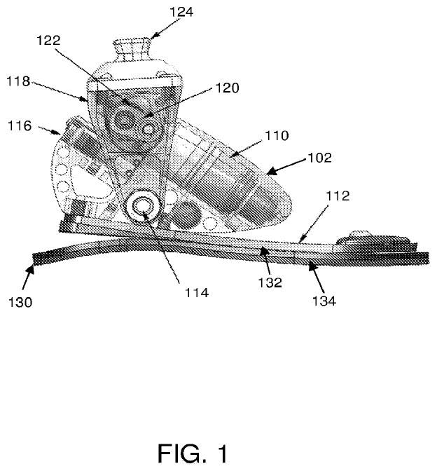

FIG. 1 illustrates elements of one configuration of the prosthetic foot/ankle system. The main housing or foot housing 102 may contain: a linear hydraulic cylinder 110, rigid mounting fastening systems for the energy storing spring elements 112, the revolute joint 114 about which the foot pivots, and manual adjustment valves 116 or electric adjustment valves. A foot support 118 attaches to the linear hydraulic cylinder 110 through linkages 120 which are connected to the linear hydraulic cylinder 110. In this example, the foot support is a clevis. The foot support 118 can also be attached to the revolute joint 114. The energy storing foot plates or energy storing springs may include a main spring 132 and/or an energy storing sole plate 134.

As the foot support 118 moves, the linkage 120 transfers that motion to the linear hydraulic cylinder 110, displacing hydraulic fluid in the linear hydraulic cylinder 110. The linkage 120 configuration helps reduce the total build height of the prosthesis. The resistances to hydraulic flow in the plantar flexion and dorsiflexion flow directions are controlled by the two independently adjustable manual adjustment valves or electric adjustment valves.

The orientation of the linear hydraulic cylinder 110 and the position of the revolute joint 114 may also improve the functionality of the prosthetic foot/ankle system. The position of the revolute joint 114 is located at a defined position with respect to the remnant limb to mimic the intact human foot/ankle. For example, the revolute joint 114 may be at an estimated position of where the amputee's intact ankle was located. An individual using this technology can ambulate (i.e., walk) with a more symmetric gait because the position of the revolute joint 114 can be located to be similar to or to match that of the primary axis of rotation of an intact ankle.

In one configuration, the horizontal distance from the heel 130 to the revolute joint 114 may be approximately one third of the overall length of the foot. The vertical distance from the ground or floor to the revolute joint 114 may be approximately one-eighth the length of the total foot length.

The stiffness of the energy storing spring elements 112 may be based on the vertical displacement of the center of pressure of an intact normal foot. The center of pressure is the position of maximum pressure on the bottom of the foot during normal walking. This center of pressure moves from the heel at heel-strike to the toe at toe-off. The shape and stiffness of the spring elements of the foot/ankle system are designed so that the center of pressure progresses from heel to toe in a way that mimics the intact foot. Furthermore, the stiffness of the foot can be designed such that the vertical deflection of the spring elements matches that of the vertical deflection of the intact foot at the center of pressure as the pressure progresses from heel to toe.

The linkages 120 shown in FIG. 1 may be oriented such that the system is able to rotate through a defined number of degrees of hydraulic motion (e.g., 5-30 degrees with 15 or 20 degrees being a useful amount of rotation for many ankles). The positioning of the linkages can be designed to limit off-axis piston shaft loading when the piston shaft is fully extended, as at foot flat, by aligning the linkage and the piston shaft axis at high loading conditions.

This foot/ankle system is also capable of heel height adjustment. This heel height adjustment may be accomplished by adjusting where the linkage 120 attaches to the linear hydraulic piston shaft. By adjusting the linkage position on the hydraulic piston shaft using the adjustment yoke or adjustable sliding yoke 122, the foot, ankle and/or housing with a heel can be pitched to the desired heel height. In one configuration, a mounting pyramid 124 can allow for user alignment of the prosthetic foot/ankle with a remnant limb of an amputee.

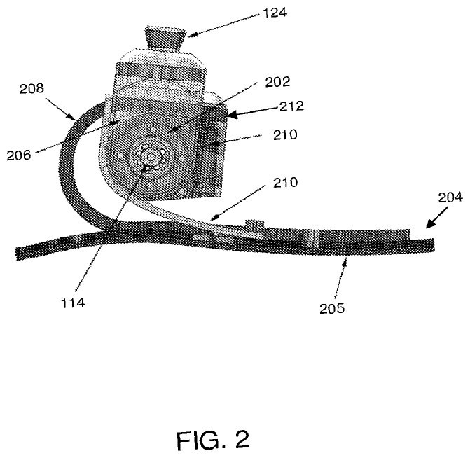

A second configuration of the prosthetic foot/ankle system will now be described that utilizes a rotary hydraulic damper 202 that is fixed solidly to spring elements 204, including an energy storing foot plate 205. FIG. 2 illustrates selected elements of the rotary foot/ankle system. The rotary hydraulic damper housing 212 is rigidly attached to the spring elements 204. The clevis 206 is attached to the rotary hydraulic damper at the revolute joint 114.

In this embodiment, the spring elements may wrap around the rotary hydraulic damper 202 to maximize the amount of spring material that can store and release energy. For example, a main spring element 208 may wrap around the rotary hydraulic damper 202. This embodiment allows for a defined number of degrees of hydraulic angular displacement (e.g., 20 degrees) and independent dorsiflexion and plantar flexion manual or electric adjustments.

Both of the configurations illustrated in FIGS. 1 and 2 have the ability to adapt to slopes and uneven surfaces. This adaptation is achieved by allowing the ankle to plantar flex when an individual using the foot puts weight on the prosthesis. Hydraulic resistance to plantar flexion can be adjusted using a manually adjustable hydraulic valve or electrically adjustable hydraulic valve. Adjusting the plantar flexion allows the user to adjust the amount of hydraulic shock absorption at heel strike. This shock absorption may also be provided by the foot/ankle toe-lift spring 210. Both energy-dissipating hydraulic impedance and the energy-storing spring elements resist plantar flexion. Both plantar flexion hydraulic impedance and the spring elements' impedances are adjustable such that the system can exhibit the desired amount of shock absorption.

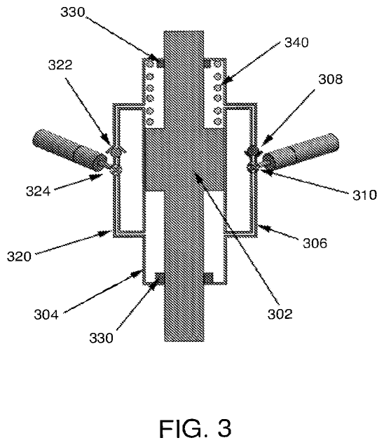

FIG. 3 illustrates a schematic of an example fluid flow configuration for the linear hydraulic cylinder 304 of the foot/ankle system. When the ankle rotates, the linear hydraulic piston 302 moves within the linear hydraulic cylinder 304. Seals 330 may help ensure the linear hydraulic cylinder 304 does not leak hydraulic fluid. When the ankle plantar flexes, the linear hydraulic piston 302 forces fluid through the plantar flexion hydraulic pathway 306 with its respective plantar flexion check valve 308 and plantar flexion resistance adjustment valve 310. The dorsiflexion resistance adjustment valve may be a manual resistance adjustment valve or an electric resistance adjustment valve. The linear hydraulic cylinder 304 may also contain an optional internal toe lift spring 340 that is within the linear hydraulic cylinder 304.

When the ankle is dorsiflexed, the linear hydraulic piston 302 in the linear hydraulic cylinder 304 forces fluid through the dorsiflexion hydraulic pathway 320 with its respective dorsiflexion check valve 322 and dorsiflexion resistance adjustment valve 324. The dorsiflexion resistance adjustment valve 324 may be a manual resistance adjustment valve or an electric resistance adjustment valve. The structure and operations that are described with respected to a linear hydraulic piston 302 and linear hydraulic cylinder 304 may also be applied to rotary hydraulic configuration.

Reference was made to the examples illustrated in the drawings, and specific language was used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1-20. (canceled)

21. A device to control movement of a prosthesis, comprising:
a foot support to provide a portion of the prosthesis;
a first joint coupled to the foot support;
a hydraulic damper coupled with the foot support via the first joint;
a second joint coupled with the hydraulic damper; and
an energy storing spring coupled with the hydraulic damper, the second joint to allow movement of the hydraulic damper relative to the energy storing spring.

22. The device of claim 21, wherein the energy storing spring is coupled with the hydraulic damper via the second joint, wherein the second joint is a revolute joint.

23. The device of claim 21, wherein the energy storing spring comprises a main spring coupled with an energy storing sole plate.

24. The device of claim 21, wherein a stiffness of the energy storing spring is based on a vertical displacement of a center of pressure of an intact normal foot.

25. The device of claim 21, wherein the energy storing spring comprises a surface that is curved based on a vertical displacement of a center of pressure of an intact normal foot.

26. The device of claim 21, further comprising a manual valve to adjust a resistance to flow in the hydraulic damper.

27. The device of claim 21, wherein the energy storing spring comprises a leaf spring.

28. The device of claim 21, wherein the hydraulic damper is a linear hydraulic damper.

29. The device of claim 21, wherein the second joint is a revolute joint at a position corresponding to an estimated position of an intact ankle.

30. A prosthetic device, comprising:
a foot support;
a hydraulic damper, the hydraulic damper is a linear hydraulic damper;
a first joint coupled with the foot support and with the hydraulic damper;
a second joint coupled with the hydraulic damper; and
an energy storing spring coupled with the hydraulic damper, the energy storing spring comprising a spring coupled with a plate.

31. The prosthetic device of claim 30, wherein the energy storing spring is coupled with the hydraulic damper via the second joint.

32. The prosthetic device of claim 30, wherein a stiffness of the energy storing spring is based on a vertical displacement of a center of pressure of an intact normal foot.

33. The prosthetic device of claim 30, further comprising a manual valve to adjust a resistance to flow in the hydraulic damper.

34. The prosthetic device of claim 30, wherein the energy storing spring comprises a leaf spring.

35. The prosthetic device of claim 30, wherein the second joint is a revolute joint at a position corresponding to an estimated position of an intact ankle.

36. The prosthetic device of claim 30, further comprising a mounting pyramid coupled with the foot support, wherein the foot support is between the mounting pyramid and the energy storing spring.

37. The prosthetic device of claim 30, wherein the plate comprises a curvature.

38. The prosthetic device of claim 30, wherein the hydraulic damper comprises a first end attached to the first joint and a second end attached to the second joint.

39. The prosthetic device of claim 30, wherein the hydraulic damper is at an angle to an axis through the foot support and the second joint.

40. The prosthetic device of claim 30, wherein the first joint is a linkage.
